(19) United States
(12) Patent Application Publication
(10) Pub. No.: US 2021/0196671 A1
(43) Pub. Date: Jul. 1, 2021

(54) COMPOSITIONS COMPRISING 3'-O-METHYL-4'-O-SULFATE EPICATECHIN AND THERAPEUTIC USES OF SUCH COMPOSITIONS

(71) Applicant: SOCIÉTÉ DES PRODUITS NESTLÉ S.A., Vevey (CH)

(72) Inventors: Lucas Actis Goretta, Lausanne 26 (CH); Amaury Paatin, Lausanne 26 (CH); Stephanie Michlig Gonzalez, Le Mont-sur-Lausanne (CH); Coline Legrand, Lausanne (CH); Susana Camacho, Lutry (CH); Johannes Le Coutre, Pully (CH)

(21) Appl. No.: 16/071,241

(22) PCT Filed: Jan. 13, 2017
(86) PCT No.: PCT/EP2017/050657

§ 371 Date: Jul. 19, 2018

Related U.S. Application Data
(60) Provisional application No. 62/287,055, filed on Jan. 26, 2016.

(51) Int. Cl. A61K 31/353; A61K 9/00; A23L 33/00; A23L 33/10

(52) U.S. Cl. CPC A61K 31/353; A61K 9/0056; A23V 2002/00; A23L 33/40; A23L 33/10; A61K 9/0019

(57) ABSTRACT

Compositions can comprise the catechin metabolite 3′-O-methyl-4′-O-sulfate epicatechin. In some embodiments, the composition can be used in a method for blood vessel dilation and/or increased delivery of blood flow to tissues in the body, for example by administering the composition to an individual having or at risk of high blood pressure or a cardiovascular disease. In some embodiments, the compositions are used for weight maintenance or weight loss. In further embodiments the compositions are used for treating or preventing gestational diabetes mellitus. The compositions may be administered orally as a food product in which the 3′-O-methyl-4′-O-sulfate epicatechinis present in a concentration of at least 0.01 mg/g of the food product.

### BACKGROUND

The present disclosure generally relates to compositions comprising a catechin metabolite. More specifically, the present disclosure relates to compositions comprising the catechin metabolite 3′-O-methyl-4′-O-sulfate epicatechin and further relates to methods comprising administering such compositions.

A condition adversely affecting some individuals is high blood pressure. Blood pressure is the force of blood pushing against the walls of the arteries as the heart pumps out blood. High blood pressure is a serious condition that is associated with a higher risk of cardiovascular diseases and can lead to, for example, coronary heart disease, heart failure, stroke, kidney failure, and other health problems.

Hypertension is a condition caused by a sustained high blood pressure. Hypertension is a cardiac chronic medical condition in which the systemic arterial blood pressure is outside a normal range. Hypertension generally refers to a condition where a systolic blood pressure is 140 mmHg or higher or a diastolic blood pressure is 90 mmHg or higher. Hypertension is classified as either primary or secondary. About 90-95% of hypertension cases are primary hypertension, which refers to high blood pressure for which no medical cause has been found. The remaining 5-10% of cases are secondary hypertension, which refers to high blood pressure caused by other conditions that affect the kidneys, arteries, heart, or endocrine system.

The incidence of hypertension is increasing all over the world. In addition, hypertension may cause fatal complications such as cerebral stroke, heart failure, and coronary artery diseases, even among minor or mild patients exhibiting no external symptoms.

Hypertension is commonly addressed with interventions that are not fully effective and thus could benefit from improved methods and compositions. High blood pressure and hypertension can also be problems during pregnancy.

### SUMMARY

Without wishing to be bound by theory, the present inventors believe that 3′-O-methyl-4′-O-sulfate epicatechin is effective to modulate nitric oxide levels to achieve blood vessel dilation and/or increased delivery of blood flow to tissues in the body. In turn, the blood vessel dilation and/or increased delivery of blood flow can reduce blood pressure, stimulate protein synthesis, improve blood circulation (for example improve blood brain circulation), increase release of growth factors, enhance immune function, and/or improve one or more of insulin sensitivity, glucose tolerance, mood, memory or cognition.

Accordingly, in a general embodiment, the present disclosure provides a method for blood vessel dilation and/or increased delivery of blood flow to tissues in the body. The method comprises administering a composition comprising 3′-O-methyl-4′-O-sulfate epicatechin to an individual.

In an embodiment, the composition is orally administered to the individual as a food product in which the 3′-O-methyl-4′-O-sulfate epicatechin is present in a concentration of at least 0.01 mg/g of the food product.

In an embodiment, the composition is administered to the individual at least once a day for at least one week.

In an embodiment, the 3′-O-methyl-4′-O-sulfate epicatechin is chemically synthesized.

In an embodiment, the individual has or is at risk of high blood pressure.

In an embodiment, the individual has or is at risk of a cardiovascular disease. In an embodiment the composition is administered to prevent preeclampsia and/or IUGR.

In an embodiment, the composition is administered to the individual in an amount that achieves a therapeutic effect selected from the group consisting of reduced blood pressure, improvement of blood circulation (for example improvement of blood brain circulation), reduction of endothelial dysfunction, stimulation of protein synthesis, increased release of growth factors, enhanced immune function, and combinations thereof, the method comprising administering to the individual a composition comprising 3′-O-methyl-4′-O-sulfate epicatechin. For example the composition may be administered to an individual in an amount that achieves a therapeutic effect selected from the group consisting of reduced blood pressure, stimulation of protein synthesis, increased release of growth factors, enhanced immune function, and combinations thereof, the method comprising administering to the individual a composition comprising 3′-O-methyl-4′-O-sulfate epicatechin.

In an embodiment, the composition is administered to the individual in an amount that improves a characteristic selected from the group consisting of insulin resistance, glucose tolerance and a combination thereof. The individual can be selected from the group consisting of an infant born preterm, an infant experiencing intrauterine growth restriction, a pregnant woman, a pregnant woman suffering from gestational diabetes or at risk of suffering from gestational diabetes mellitus (GDM), a human suffering from insulin resistance, a human suffering from impaired glucose tolerance, and a human suffering from type II diabetes.

In an embodiment, the composition is administered to the individual in an amount that improves one or more of cognitive performance, cognition, mood, or memory. The individual can have a condition selected from the group consisting of cognitive decline, cognitive aging, mild cognitive impairment, dementia, a mood disorder, memory loss, and combinations thereof. For example, the individual can be an elderly human having cognitive aging.

In an embodiment, the composition is injected into the individual.

In another embodiment, a method for weight maintenance or weight loss is provided. The method comprises administering to an individual a composition comprising 3′-O-methyl-4′-O-sulfate epicatechin. The composition can be orally administered to the individual as a food product in which the 3′-O-methyl-4′-O-sulfate epicatechin is present in a concentration of at least 0.01 mg/g of the food product and/or can be administered to the individual at least once a day for at least one week. In an embodiment, the 3′-O-methyl-4′-O-sulfate epicatechin is chemically synthesized. The composition can be injected into the individual.

In an embodiment, the composition further comprises an additional ingredient in an amount effective to promote weight maintenance or weight loss.

In an embodiment, the composition is administered to provide an amount of the 3′-O-methyl-4′-O-sulfate epicatechin that increases at least one characteristic selected from the group consisting of energy expenditure, sympathetic nervous system activity, and fat oxidation.

In an embodiment, the individual has or is at risk of obesity or overweight.

In an embodiment, the individual is participating in a weight loss program selected from the group consisting of a low-fat diet, a low-carbohydrate diet, a low-calorie diet, a very low-calorie diet, endurance training, strength training, and combinations thereof.

In another embodiment, the present disclosure provides a method for treating or preventing endothelium dysfunction comprising administering to an individual in need thereof or at risk thereof a composition comprising 3′-O-methyl-4′-O-sulfate epicatechin. The composition can be orally administered to the individual as a food product in which the 3′-O-methyl-4′-O-sulfate epicatechin is present in a concentration of at least 0.01 mg/g of the food product. It can be ingested in a special form to prevent degradation in the stomach. The composition can be administered to the individual at least once a day for at least one week.

An advantage of the present disclosure is to use a compound that is easily consumed.

Another advantage of the present disclosure is to provide tolerable side effects or no side effects while achieving blood vessel dilation and/or increased delivery of blood flow to tissues in the body.

Yet another advantage of the present disclosure is to use a compound that has increased acceptability, reduced pungency, and improved tolerance in the gastrointestinal tract relative to capsaicin.

Still another advantage of the present disclosure is to treat or prevent high blood pressure.

An additional advantage of the present disclosure is to treat or prevent cardiovascular diseases.

Additional features and advantages are described herein, and will be apparent from, the following Detailed Description and the FIGURES.

### BRIEF DESCRIPTION OF THE FIGURES

FIG. 1 shows the structure of sodium 3′-O-methyl-4′-O-sulfate epicatechin.

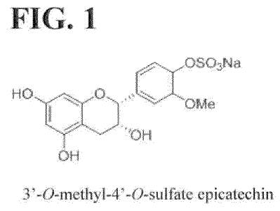

### DETAILED DESCRIPTION

All percentages expressed herein are by weight of the total weight of the composition unless expressed otherwise. When reference is made to the pH, values correspond to pH measured at 25° C. with standard equipment. As used in this disclosure and the appended claims, the singular forms “a,” “an” and “the” include plural referents unless the context clearly dictates otherwise. As used herein, “about” is understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, all numerical ranges herein should be understood to include all integers, whole or fractions, within the range. The compositions disclosed herein may lack any element that is not specifically disclosed herein. Thus, a disclosure of an embodiment using the term “comprising” includes a disclosure of embodiments “consisting essentially of” and “consisting of” the components identified.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

For adults, “high blood pressure” is a systolic blood pressure of 140 mmHg or higher and/or a diastolic blood pressure of 90 mmHg or higher. High blood pressure includes both primary and secondary hypertension. Non-limiting examples of individuals “at risk” of high blood pressure include humans of age 60 or older, overweight or obese individuals, individuals who smoke at least once a day, humans who consume at least 2.4 g of sodium each day, humans who consume less than 4.7 g of potassium each day, individuals who perform aerobic exercise less than 3 days per week, men who consume more than 3 units of alcohol per day, women who consume more than 2 units of alcohol per day, individuals who have a mother or father with high blood pressure, and individuals having either (i) a systolic blood pressure from 120 to 139 mmHg and a diastolic blood pressure from 40 to 80 mmHg or (ii) a systolic blood pressure from 70 to 140 mmHg and a diastolic blood pressure from 80 to 89 mmHg.

Cardiovascular diseases are diseases associated with high blood pressure.

Non-limiting examples of cardiovascular diseases include coronary heart disease, heart failure, peripheral arterial disease, hypertensive retinopathy, hypertensive encephalopathy, stroke, kidney failure, gestational hypertension, pre-eclampsia, eclampsia, and combinations thereof. Non-limiting examples of individuals “at risk” of a cardiovascular disease include individuals with high blood pressure, individuals at risk of high blood pressure, and individuals with high blood cholesterol (e.g., total cholesterol of 240 mg/dL or greater and/or LDL (low-density lipoprotein) of 160 mg/dL or greater), diabetes, and overweight or obesity. On a population basis, a reduction of 2 mm Hg in diastolic blood pressure is estimated to result in a 15% reduction in risk of stroke and a 6% reduction in risk of coronary heart disease.

As used herein, an “effective amount” is an amount that prevents a deficiency, treats a disease or medical condition in an individual or, more generally, reduces symptoms, manages progression of the diseases or provides a nutritional, physiological, or medical benefit to the individual. The relative terms “improved,” “increased,” “enhanced” and the like refer to the effects of the composition comprising 3′-O-methyl-4′-O-sulfate epicatechin (disclosed herein) relative to a composition lacking 3′-O-methyl-4′-O-sulfate epicatechin but otherwise identical.

“Animal” includes, but is not limited to, mammals, which includes but is not limited to, rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “patient” is understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human that can benefit from the treatment. Said individual may be planning to be pregnant, or pregnant and/or lactating.

“Overweight” is defined for a human as a BMI between 25 and 30. “Obese” is defined for a human as a BMI greater than 30. “Weight loss” is a reduction of the total body weight. Weight loss may, for example, refer to the loss of total body mass in an effort to improve fitness, health, and/or appearance. “Weight management” or “weight maintenance” relates to maintaining a total body weight. For example, weight management may relate to maintaining a BMI in the area of 18.5-25 which is considered to be normal.

The term “GDM” as used herein refers to any degree of impaired glucose tolerance that onsets or is first recognized during pregnancy. The term “preeclampsia” as used herein refers to a pregnancy condition diagnosed by high blood pressure and one or more of the following complications after the 20^{th} week of pregnancy: protein in urine, a low platelet count, impaired liver function, signs of kidney trouble other than protein in urine, fluid in the lungs (pulmonary edema), visual disturbances, new (onset) headaches. The term IUGR is a condition wherein the growth of an individual's foetus/baby is compromised and restricted in-utero so that said foetus/baby is smaller for gestational age. IUGR may result in said foetus/baby being a low birth weight baby.

As set forth above, the present inventors believe that 3′-O-methyl-4′-O-sulfate epicatechin can be used in methods that modulate nitric oxide (NO) levels. NO is important for relaxation of blood vessels and delivery of blood flow to tissues in the body. With improved blood flow, nutrients and other compounds in the blood can be delivered more efficiently to the skeletal muscle tissues. Furthermore, NO is an anabolic signal as well as a facilitator for stimulation of protein synthesis and release of growth factors such as polyamines. NO also leads to release of insulin and IGF-1, leading to increased uptake of anabolic substrates and also bio-utilization of the substrates. NO is also involved in immune function via T-cell stimulation. It has been suggested that aberrant NO synthesis and/or blood flow in the uterus and/or placenta may contribute to the pathophysiologic changes seen in preeclampsia and IUGR. Preeclampsia is a leading cause of maternal death throughout the world and is responsible for significant baby morbidity and mortality. Furthermore, preeclampsia has healthcare implications for the women later in life with those having suffered having an increased risk of hypertension, coronary artery disease, stroke and type 2 diabetes. IUGR is a major cause of perinatal and neonatal mortality and morbidity and is associated with an increased risk of several health problems in later life for the foetus/baby e.g. an increased risk of cardio vascular disease and type II diabetes.

Accordingly, an aspect of the present disclosure is a method for blood vessel dilation and/or increased delivery of blood flow to tissues in the body comprising administering a composition comprising 3′-O-methyl-4′-O-sulfate epicatechin to an individual. The composition can be administered to an individual at least once a day for at least one week, preferably for at least one month. The composition may be administered pre-pregnancy and in the case of a pregnant individual the composition can be administered for any period of pregnancy.

The composition can be administered to an individual having or at risk of high blood pressure and/or a cardiovascular disease and/or Preeclampsia and/or IUGR. In an embodiment, the composition is administered to the individual to provide an amount of the 3′-O-methyl-4′-O-sulfate epicatechin that achieves a therapeutic effect selected from the group consisting of reduced blood pressure, stimulation of protein synthesis, improvement of blood circulation, improvement of blood circulation in the brain, increased release of growth factors, enhanced immune function, and combinations thereof, the method comprising administering to the individual a composition comprising 3′-O-methyl-4′-O-sulfate epicatechin. In some embodiments, the increased blood flow from the 3′-O-methyl-4′-O-sulfate epicatechin can be used to treat or prevent erectile dysfunction, or placental insufficiency and/or a condition associated therewith. Non limiting examples of conditions associated with placental insufficiency include: non optimal fetal development, and birth defects, in particular after 20 weeks of gestation. In some embodiments, the increased blood flow from the 3′-O-methyl-4′-O-sulfate epicatechin can be used in a pregnant woman to ensure an optimum supply of nutrients to her developing fetus, or it can be used in a woman postpartum during lactation to ensure an optimum supply of nutrients to her lactating offspring. With respect to preeclampsia and/or IUGR, the composition can be administered for any period of pregnancy, or pre-pregnancy when a woman is trying to get pregnant. Since preeclampsia is most likely to occur in the 3^{rd} trimesters of pregnancy it may be beneficial if the composition is administered in the 3^{rd} trimester of pregnancy.

In an embodiment, the composition is administered to the individual to provide an amount of the 3′-O-methyl-4′-O-sulfate epicatechin that improves a characteristic selected from the group consisting of insulin resistance, glucose tolerance and a combination thereof. The composition can thereby reduce glycemia. In such embodiments, the individual can be an infant (a child under the age of 12 months) born preterm and/or experiencing intrauterine growth restriction (IUGR), a pregnant woman suffering from or at risk of suffering from gestational diabetes mellitus (GDM) or a lactating woman; or a child (up to twelve years of age), an adolescent (twelve to eighteen years of age), or an adult (over eighteen years of age) suffering from insulin resistance and/or type II diabetes, such as an animal such as a human. In an embodiment, the method can comprise identifying the individual as in need of an improvement in insulin sensitivity and/or glucose tolerance. Gestational Diabetes Mellitus (GDM) is a condition specifically affecting pregnant women, it is a condition that can increase the risk of a number of maternal-fetal conditions, including macrosomia, birth injury, shoulder dystocia, premature delivery, and caesarian delivery. Mothers suffering from GDM also have an increased risk of developing type II diabetes immediately after pregnancy and later in life. Also, the foetus/infant of mothers suffering from GDM have an increased risk of developing an impaired glucose tolerance and/or suffering from excess weight/adiposity and associated metabolic disorders e.g. type II diabetes and obesity. With respect to GDM the composition can be administered for any period of pregnancy, or pre pregnancy when a woman is trying to get pregnant. Since GDM is most likely to occur in the 2^{nd} and 3^{rd} trimesters it may be beneficial if the composition is administered in the 2^{nd} and/or 3^{rd} trimester of pregnancy.

There is a direct link between glucose tolerance and mood, memory and cognition. For example, in a study in which participants were given an oral glucose tolerance test and cognitive tests, the older age group showed that those with poorer glucose tolerance forgot more words and had slower decision times; and, in those participants with poor glucose tolerance, a tendency for blood glucose to fall below baseline values was associated with better mood and faster working memory. Therefore, without being bound by theory, the inventors believe that 3′-O-methyl-4′-O-sulfate epicatechin can modulate blood flow to enhance insulin sensitivity and/or glucose tolerance, which in turn can improve one or more of mood, memory or cognition.

Accordingly, in an embodiment, the composition can be administered to provide an amount of 3′-O-methyl-4′-O-sulfate epicatechin that improves one or more of cognitive performance, cognition, mood or memory in an individual. The composition can treat or prevent one or more of cognitive decline, mild cognitive impairment, dementia, a mood disorder, or memory loss in an individual having or at risk of having one or more of these conditions. The method can comprise identifying the individual as having one or more of cognitive decline, mild cognitive impairment, dementia, a mood disorder, or memory loss, e.g., before initial administration of the composition. The method can comprise identifying the individual as being in need of an improvement in one or more of cognitive performance, cognition, mood or memory, e.g., before initial administration of the composition.

The composition can be administered to an infant (a child under the age of twelve months), a child (up to twelve years of age), an adolescent (twelve to eighteen years of age), an adult (over eighteen years of age), or an elderly individual (past the first two thirds of the average expected lifespan in its country of origin, preferably past the first three quarters of the average expected lifespan in its country of origin; an elderly human is a person with a chronological age of 65 years or older). The composition may be particularly effective against cognitive aging, and thus this embodiment of the method preferably comprises administration to an elderly human.

Cognitive performance may be expressed as ability and speed of learning, ability and speed of solving intellectual problems, ability to form and recall memories, reaction time, and the like. Cognition is understood as mental processes such as comprehension, inference, decision-making, planning, learning, memory, association, concept formation, language, attention, perception, action, problem solving and mental images. Cognitive decline may manifest as reduced memory; forgetfulness; word or name-finding problems; and/or decline in memory, concentration, ability to plan or organize, ability to perform complex tasks, and/or cognitive performance; and may result from age, stress, disease, or other grounds. Cognitive impairment may manifest in one or more of short-term memory loss, diminished capacity to learn, diminished rate of learning, or diminished attention.

The term “mood” refers to a state or quality of feeling (an emotional state) at a particular time. Moods differ from simple emotions in that they are less specific, less intense, and less likely to be triggered by a particular stimulus or event. Moods generally have either a positive or negative valence. An improved mood may comprise one or more of a decreased anxiety level, a decreased stress level, an increased perceived energy level, or a more positive emotional state.

In another aspect of the present disclosure, the composition comprising 3′-O-methyl-4′-O-sulfate epicatechin can be used in a method to support weight management or promote weight loss. For example, the composition can be administered to an individual, such as a mammal, that is managing their weight or undergoing a weight loss program. The weight loss program may include, for example, a weight loss diet (e.g., one or more of a low-fat diet, for example a diet with less than 20% of the calories from fat, preferably less than 15% from fat; a low-carbohydrate diet, for example a diet with less than 20% of the calories from carbohydrates; a low-calorie diet, for example a diet with less calories per day relative to the individual's previous intake before the diet, or a diet with less calories per day relative to an average person of similar body type; or a very low-calorie diet, for example a diet with 800 kcal (3,300 kJ) per day or less). Additionally or alternatively, the weight loss program may include a weight loss training regimen (e.g. endurance and/or strength training).

The individual can be overweight or obese or at risk thereof. The composition can also comprise an additional weight loss ingredient. The method can comprise identifying the individual as being in need of weight management or weight loss and/or identifying the individual as obese or overweight, e.g., before initial administration of the composition. The composition can be administered to provide an amount of the 3′-O-methyl-4′-O-sulfate epicatechin that increases at least one characteristic selected from the group consisting of energy expenditure, sympathetic nervous system activity, and fat oxidation.

In any of the methods disclosed herein, the composition can comprise an extract enriched with 3′-O-methyl-4′-O-sulfate epicatechin.

In any of the methods disclosed herein, the composition can be a medical food, a nutritional composition, a pharmaceutical, a food composition or a pet food composition.

In any of the methods disclosed herein, the composition may be administered to the individual at least once a day for at least one week.

In any of the methods disclosed herein, the 3′-O-methyl-4′-O-sulfate epicatechin may be chemically synthesized.

In an embodiment, the 3′-O-methyl-4′-O-sulfate epicatechin can be synthesized. For example, the 3′-O-methyl-4′-O-sulfate epicatechin can be synthesized as disclosed in Zhang et al. 2013 (Mingbao Zhang, G. Erik Jagdmann, Jr., Michael Van Zandt, Ryan Sheeler, Paul Beckett and Hagen Schroeter, Chemical Synthesis and Characterization of Epicatechin Glucuronides and Sulfates: Bioanalytical Standards for Epicatechin Metabolite Identification, J. Nat. Prod., 76, 157-169 (2013)).

The 3′-O-methyl-4′-O-sulfate epicatechin can be co-administered with a polyphenol to increase the bioavailability of the 3′-O-methyl-4′-O-sulfate epicatechin, as disclosed in WO2014/083172. For example, the composition can comprise 3′-O-methyl-4′-O-sulfate epicatechin and at least one polyphenolic compound selected from the group consisting of flavonols, flavones, isoflavones, flavanones, and combinations thereof. In a preferred embodiment, a flavone and/or flavanone is selected from the group consisting of isorhamnetin, kaempfernol, diosmetin, nevadensin, chrysin, hesperitin, and combinations thereof.

The composition comprising 3′-O-methyl-4′-O-sulfate epicatechin may be a medicament, a food product, a medical food, a functional food, an oral nutritional supplement, a nutritional composition, an oral cosmetic, or a supplement to a food product and is preferably orally administered. A medical food product is specially formulated and intended for the dietary management of diseases or medical conditions (e.g., prevent or treat diseases or undesirable medical conditions). A medical food product can provide clinical nutrition, for example fulfilling special nutritional needs of patients with a medical condition or other persons with specific nutritional needs. A medical food product can be in the form of a complete meal, part of a meal, as a food additive, or a powder for dissolution. The compositions may be for maternal use prenatally, during pregnancy and/or during lactation, in particular they may be of use during pregnancy at 20 weeks of gestation and thereafter.

In an embodiment, the composition is administered orally as a food product in which the 3′-O-methyl-4′-O-sulfate epicatechin is present in a concentration of at least 0.01 mg/g of the food product, preferably at least 0.1 mg/g of the food product, more preferably at least 1 mg/g of the food product, even more preferably 10 mg/g of the food product, or more.

A food product, medical food or nutritional composition includes any number of optional additional ingredients, including conventional food additives, for example one or more proteins, carbohydrates, fats, vitamins, minerals, acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers and/or vitamins. The optional ingredients can be added in any suitable amount.

A food product, medical food or nutritional composition can be in any oral nutritional form, e.g. as a health drink, as a ready-made drink, optionally as a soft drink, including juices, milk-shake, yogurt drink, smoothie or soy-based drink, in a bar, or dispersed in foods of any sort, such as baked products, cereal bars, dairy bars, snack-foods, soups, breakfast cereals, muesli, candies, tabs, cookies, biscuits, crackers (such as a rice crackers), and dairy products.

A supplement may be in the form of tablets, capsules, pastilles or a liquid, for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins or the like), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The supplement may for example be a maternal supplement for administration to an individual pre pregnancy when said individual is trying to get pregnant, and/or during pregnancy and/or during lactation.

The supplement can be added in a product acceptable to the consumer as an ingestible carrier or support. Non-limiting examples of such carriers or supports are a pharmaceutical, a food composition, and a pet food composition. Non-limiting examples for food and pet food compositions are milks, yogurts, curds, cheeses, fermented milks, milk-based fermented products, fermented cereal based products, milk-based powders, human milks, preterm formulas, infant formulas, oral supplements, and tube feedings.

In some embodiments, the composition comprising 3′-O-methyl-4′-O-sulfate epicatechin is in an injectable form, and the methods disclosed herein can comprise injecting the individual with the composition.

### EXAMPLE

The following non-limiting example presents scientific data developing and supporting the concept of 3′-O-methyl-4′-O-sulfate epicatechin acting as an agonist of TRPA1.

Several epicatechin metabolites were tested in vitro to determine if any of these compounds are an agonist of the cation channel of TRPA1. Specifically, the activation of CHO cells expressing h-TRPA1 was tested according to the calcium imaging method described in Riera et al. (Riera, C. E., Vogel, H., Simon, S. A., Damak, S., & le Coutre, J. Sensory attributes of complex tasting divalent salts are mediated by TRPM5 and TRPV1 channels. J. Neurosci. 29, 2654-2662 (2009)).

Epicatechin metabolites were tested and results are summarized in Table 1 below.

TABLE 1

| | Concentration | | Activity vs | |
|---|---|---|---|---|
| Compounds | (Max) tested | | hTRPA1 | EC50 |
| 3′-O-sulfate-EC | 100 | mM | Inactive | |
| 4′-O-sulfate-EC | 100 | mM | Inactive | |
| 3′-O-methyl-EC | 100 | mM | Inactive | |
| 4′-O-methyl-EC | 100 | mM | Inactive | |
| 3′-O-methyl-7-O-sulfate-EC | 100 | mM | Inactive | |
| 3′-O-methyl-4′-O-sulfate-EC | 1 | mM | Active | ~200 mM |
| Hesperetin-3-O-Glucuronide | 3 | mM | Inactive | |
| Hesperetin-4-O-Glucuronide | 3 | mM | Inactive | |
| Hesperetin-7-3-O-Glucuronide | 3 | mM | Inactive | |

Further aspects and embodiments of the invention are set out in the following lettered paragraphs:
A. A method for blood vessel dilation and/or increased delivery of blood flow to tissues in the body, the method comprising administering a composition comprising 3′-O-methyl-4′-O-sulfate epicatechin to an individual.
B. The method according to paragraph A wherein the individual has or is at risk of high blood pressure or a cardiovascular disease.
C. The method according to paragraph A or paragraph B wherein the composition is administered to the individual in an amount that achieves a therapeutic effect selected from the group consisting of reduced blood pressure, improvement of blood circulation, improvement of blood brain circulation, reduction of endothelial dysfunction, stimulation of protein synthesis, increased release of growth factors, enhanced immune function, and combinations thereof.
D. The method according to paragraph A wherein the composition is administered to the individual in an amount that improves a characteristic selected from the group consisting of insulin resistance, glucose tolerance and a combination thereof.
E. The method according to paragraph D wherein the individual is selected from the group consisting of an infant born preterm, an infant experiencing intrauterine growth restriction, a pregnant woman suffering from gestational diabetes, gestational hypertension, pre-eclampsia, eclampsia, and/or placental insufficiency, a human suffering from insulin resistance, a human suffering from impaired glucose tolerance, and a human suffering from type II diabetes.
F. The method according to paragraph A wherein the composition is administered to the individual in an amount that improves one or more of cognitive performance, cognition, mood, or memory.
G. The method according to paragraph F wherein the individual has a condition selected from the group consisting of cognitive decline, mild cognitive impairment, dementia, a mood disorder, memory loss, and combinations thereof.
H. The method according to paragraph F or paragraph G wherein the individual is an elderly human having cognitive aging.
I. A method for weight maintenance or weight loss comprising administering a composition comprising 3′-O-methyl-4′-O-sulfate epicatechin to an individual.
J. The method according to paragraph I wherein the composition further comprises an additional ingredient in an amount effective to promote weight maintenance or weight loss.
K. The method according to paragraph I or paragraph J wherein the composition is administered to provide an amount of the 3′-O-methyl-4′-O-sulfate epicatechin that increases at least one characteristic selected from the group consisting of energy expenditure, sympathetic nervous system activity, and fat oxidation.
L. The method according to any one of paragraphs I to K wherein the individual has or is at risk of obesity or overweight.
M. A method for treating or preventing endothelium dysfunction comprising administering to an individual in need thereof or at risk thereof a composition comprising 3′-O-methyl-4′-O-sulfate epicatechin.
N. A method for treating or preventing preeclampsia and/or IUGR comprising administering to an individual in need thereof or at risk thereof a composition comprising 3′-O-methyl-4′-O-sulfate epicatechin.
O. A method for treating or preventing gestational diabetes mellitus comprising administering to an individual in need thereof or at risk thereof a composition comprising 3′-O-methyl-4′-O-sulfate epicatechin.
P. The method according to paragraph N or paragraph 0 wherein the composition is administered to the individual pre-pregnancy when said individual is trying to get pregnant and/or, during pregnancy, and or during lactation.
Q. The method according to any one of the preceding paragraphs wherein the composition is injected into the individual.
R. The method according to any one of paragraphs A to P wherein the composition is orally administered to the individual as a food product in which the 3′-O-methyl-4′-O-sulfate epicatechin is present in a concentration of at least 0.01 mg/g of the food product.
S. The method according to any one of paragraphs N to P wherein the composition is orally administered to the individual as a maternal supplement in which the 3′-O-methyl-4′-O-sulfate epicatechin is present in a concentration of at least 0.01 mg/g of the composition.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for blood vessel dilation and/or increased delivery of blood flow to tissues in the body, the method comprising administering a composition comprising 3′-O-methyl-4′-O-sulfate epicatechin to an individual in need of same.

2. The method of claim 1 wherein the composition is orally administered to the individual as a food product in which the 3′-O-methyl-4′-O-sulfate epicatechin is present in a concentration of at least 0.01 mg/g of the food product.

3. The method of claim 1 wherein the composition is administered to the individual at least once a day for at least one week.

4-6. (canceled)

7. The method of claim 1 wherein the composition is administered to the individual in an amount that achieves a therapeutic effect selected from the group consisting of reduced blood pressure, improvement of blood circulation, improvement of blood brain circulation, reduction of endothelial dysfunction, stimulation of protein synthesis, increased release of growth factors, enhanced immune function, and combinations thereof.

8. The method of claim 1 wherein the composition is administered to the individual in an amount that improves a characteristic selected from the group consisting of insulin resistance, glucose tolerance and a combination thereof.

9. The method of claim 8 wherein the individual is selected from the group consisting of an infant born preterm, an infant experiencing intrauterine growth restriction, a pregnant woman suffering from gestational diabetes, gestational hypertension, pre-eclampsia, eclampsia, and/or placental insufficiency, a human suffering from insulin resistance, a human suffering from impaired glucose tolerance, and a human suffering from type II diabetes.

10. The method of claim 1 wherein the composition is administered to the individual in an amount that improves one or more of cognitive performance, cognition, mood, or memory.

11. The method of claim 10 wherein the individual has a condition selected from the group consisting of cognitive decline, mild cognitive impairment, dementia, a mood disorder, memory loss, and combinations thereof.

12. The method of claim 10 wherein the individual is an elderly human having cognitive aging.

13. The method of claim 1 wherein the composition is injected into the individual.

14. A method for weight maintenance or weight loss comprising administering a composition comprising 3′-O-methyl-4′-O-sulfate epicatechin to an individual.

15. The method of claim 14 wherein the composition is orally administered to the individual as a food product in which the 3′-O-methyl-4′-O-sulfate epicatechin is present in a concentration of at least 0.01 mg/g of the food product.

16. The method of claim 14 wherein the composition is administered to the individual at least once a day for at least one week.

17. (canceled)

18. The method of claim 14 wherein the composition further comprises an additional ingredient in an amount effective to promote weight maintenance or weight loss.

19. The method of claim 14 wherein the composition is administered to provide an amount of the 3′-O-methyl-4′-O-sulfate epicatechin that increases at least one characteristic selected from the group consisting of energy expenditure, sympathetic nervous system activity, and fat oxidation.

20. The method of claim 14 wherein the individual has or is at risk of obesity or overweight.

21. The method of claim 14 wherein the individual is participating in a weight loss program selected from the group consisting of a low-fat diet, a low-carbohydrate diet, a low-calorie diet, a very low-calorie diet, endurance training, strength training, and combinations thereof.

22. The method of claim 14 wherein the composition is injected into the individual.

23-28. (canceled)

29. A method for treating or preventing gestational diabetes mellitus comprising administering to an individual in need thereof or at risk thereof a composition comprising 3′-O-methyl-4′-O-sulfate epicatechin.

30. The method of claim 29 wherein the composition is orally administered to the individual as a maternal supplement in which the 3′-O-methyl-4′-O-sulfate epicatechin is present in a concentration of at least 0.01 mg/g of the composition.

31. The method of claim 29 wherein the composition is administered to the individual pre-pregnancy when said individual is trying to get pregnant and/or, during pregnancy, and or during lactation.

32. The method according to claim 29 wherein the composition is in the form of an extract enriched with 3′-O-methyl-4′-O-sulfate epicatechin.

33. (canceled)
